(19) United States
(12) Patent Application Publication
(10) Pub. No.: US 2024/0325263 A1
(43) Pub. Date: Oct. 3, 2024

(54) FLUORIDE-CONTAINING BIOACTIVE GLASSES

(71) Applicant: CORNING INCORPORATED, CORNING, NY (US)

(72) Inventors: Qiang Fu, Painted Post, NY (US); Charisse Marye Spier, Horseheads, NY (US); Nicholas Michael Stone-Weiss, Corning, NY (US)

(21) Appl. No.: 18/613,863

(22) Filed: Mar. 22, 2024

Related U.S. Application Data
(60) Provisional application No. 63/455,457, filed on Mar. 29, 2023.

(51) Int. Cl. A61K 8/25; A61K 8/19; A61K 8/21; A61K 8/28; A61Q 11/00

(52) U.S. Cl. CPC A61K 8/25; A61K 8/19; A61K 8/21; A61K 8/28; A61Q 11/00; A61K 2800/28; A61K 2800/30

(57) ABSTRACT

Glass compositions comprising 15-50 wt. % SiO₂, 25-60 wt. % CaO, 3-30 wt. % P₂O₅, 0.5-15 wt. % ZrO₂, and >0-5 wt. % F, wherein, when subjected to artificial saliva, the glass composition forms a bioactive crystalline phase, such as apatite. A matrix comprising the glass composition, wherein the glass composition is attached to the matrix or mixed therein, in which the matrix includes at least one of: a toothpaste, mouthwash, gel, varnish, rinse, spray, ointment, salve, cream, bandage, polymer film, oral formulation, pill, capsule, or transdermal formulation. The matrix may be a dentifrice composition. A method comprising applying the matrix to a tooth, in which the method remineralizes enamel of the tooth, treats caries of the tooth, treats dentin hypersensitivity of the tooth, or any combination thereof.

### CROSS-REFERENCE TO RELATED APPLICATIONS

This application claims the benefit of priority of U.S. Provisional Application Ser. No. 63/455,457, filed on Mar. 29, 2023, the content of which is relied upon and incorporated herein by reference in its entirety.

### FIELD

This disclosure generally relates to glass compositions, and more particularly to bioactive glass compositions for consumer, dental, and cosmetic applications.

### BACKGROUND

Bioactive glasses are a group of glass and/or glass ceramic materials that have shown biocompatibility or bioactivity, which has allowed them to be incorporated into human or animal physiology. Generally speaking, bioactive glasses may be able to bond with hard and soft tissues, thereby fostering growth of bone and cartilage cells. Moreover, bioactive glasses may also enable release of ions which activate expression of osteogenic genes and stimulate angiogenesis, as well as promote vascularization, wound healing, and cardiac, lung, nerve, gastrointestinal, urinary tract, and laryngeal tissue repair.

Some bioactive glasses, such as 45S5 Bioglass® sold by Haleon under the Sensodyne® brand featuring NovaMin® technology, are being investigated for their potential ability to mineralize teeth; however, the low chemical durability of these traditional bioactive glasses is problematic for compositions requiring prolonged shelf times in aqueous environments. For example, 45S5 Bioglass® requires development of a non-aqueous environment for glass particulates to be used in toothpaste applications. In addition, the speed at which 45S5 Bioglass® may mineralize teeth also is not ideal. Thus, there is a need for improved bioactive glass compositions.

### SUMMARY

In some aspects, disclosed are glass compositions comprising:
15-50 wt. % SiO₂,
25-60 wt. % CaO,
3-30 wt. % P₂O₅,
0.5-15 wt. % ZrO₂, and
>0-5 wt. % F,
wherein, when subjected to artificial saliva of Table 1A or Table 1B, the glass composition forms a bioactive crystalline phase. In some aspects, the bioactive crystalline phase forms within 14 days when subjected to artificial saliva of Table 1A or Table 1B.

In some aspects, disclosed is a matrix comprising the glass compositions disclosed herein, wherein:
the glass composition is attached to the matrix or mixed therein, and
the matrix includes at least one of: a toothpaste, mouthwash, gel, varnish, rinse, spray, ointment, salve, cream, bandage, polymer film, oral formulation, pill, capsule, or transdermal formulation.

In some aspects, a matrix comprising the glass composition is substantially free of, or does not include, a fluoride ion source (i.e., beyond the F contained within the glass composition itself), since, in some aspects, the glass composition forms a bioactive crystalline phase when subject to artificial saliva in the absence of an external fluoride ion source. However, in some aspects, it still may be desirable for a matrix include such an external fluoride ion source in addition to the glass composition (i.e., which generally already contains F⁻).

In some aspects, the matrix is a dentifrice composition comprising:
>0-20 wt. % of the glass composition, based on total weight of the matrix,
water and/or glycerin, and
one or more of a humectant, a fluoride ion source, a flavoring, a surfactant, a sweetener, a whitening agent, an abrasive, or any combination thereof.

In some aspects, disclosed is a method for making the matrix comprising combining the glass composition with a carrier (e.g., the carrier effectively may be one or more, or all, of the components of the matrix except for the glass composition).

In some aspects, disclosed is a method comprising applying to a bone or tooth the matrix. In some aspects, such method remineralizes bone, remineralizes a tooth, remineralizes enamel of a tooth, treats caries of the tooth, treats dentin hypersensitivity of the tooth, or any combination thereof.

In some aspects, disclosed is a method for making the glass composition, comprising:
combining each component of the glass composition to form a mixture,
heating the mixture to a temperature of 1500° C. or less to form a melted mixture, and
cooling the melted mixture to form a cooled mixture.

Additional features and advantages of the disclosure will be set forth in the detailed description which follows, and in part will be readily apparent to those skilled in the art from that description or recognized by practicing the aspects as described herein, including the detailed description which follows, the claims, as well as the appended drawings.

It is to be understood that both the foregoing general description and the following detailed description are merely exemplary and are intended to provide an overview or framework for understanding the nature and character of the disclosure and claims. The accompanying drawings are included to provide a further understanding of the disclosure and are incorporated into and constitute a part of this specification. The drawings illustrate various aspects of the disclosure and together with the description serve to explain the principles and operations of the various aspects.

### BRIEF DESCRIPTION OF THE DRAWINGS

The following detailed description can be further understood when read in conjunction with the following drawings. Whenever possible, the same reference numerals will be used throughout the drawings to refer to the same or like parts. It is to be understood that the figures are not drawn to scale and the size of each depicted component or the relative size of one component to another is not intended to be limiting.

FIG. 1 is a powder X-ray diffraction (XRD) spectrum illustrating apatite formation after immersion in artificial saliva for 14 days of glass compositions prepared in accordance with the disclosures herein.

FIG. 2 is a powder XRD spectrum illustrating no apatite formation of a comparative glass composition after immersion in artificial saliva for 61 days.

### DETAILED DESCRIPTION

In the following description, whenever a group is described as comprising at least one of a group of elements and combinations thereof, it is understood that the group may comprise, consist essentially of, or consist of any number of those elements recited, either individually or in combination with each other. Similarly, whenever a group is described as consisting of at least one of a group of elements or combinations thereof, it is understood that the group may consist of any number of those elements recited, either individually or in combination with each other.

Where a range of numerical values is recited herein, comprising upper and lower values, unless otherwise stated in specific circumstances, the range is intended to include the endpoints thereof, and all integers and fractions within the range. Further, when an amount, concentration, or other value or parameter is given as a range, one or more ranges, or a list of upper values and lower values, this is to be understood as specifically disclosing all ranges formed from any pair of any upper range limit or value and any lower range limit or value, regardless of whether such pairs are separately disclosed.

If the term “about” is used in describing a value or an end-point of a range, the disclosure should be understood to include the specific value or end-point referred to. As used herein, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. It is noted that the terms “substantially” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. Thus, for example, a glass that is “free” or “essentially free” or “substantially free” of Al₂O₃ (or any other component) is one in which Al₂O₃ (or any other component) is not actively added or batched into the glass, but may be present in very small amounts as a contaminant (e.g., 500, 400, 300, 200, or 100 ppm or less).

As used herein, a glass, glass-ceramic, or crystalline phase that is “bioactive” means that the glass, glass-ceramic, or crystalline phase is biologically compatible with bone, teeth, and/or tissue. By way of illustration, in some aspects, “bioactive” in this context refers to (1) the ability to form apatite (or other bioactive crystalline phases such as hydroxyapatite, fluorapatite, carbonated apatite, or any combination thereof) in a simulated body fluid, such as artificial saliva, according to ASTM F1538-03 (2017), and/or (2) the capability of binding with a desired biological material (e.g., bones, teeth, and/or tissue). Generally, there is also an absence of toxicity or other significant negative effects in a biological environment (e.g., bones, teeth, and/or tissue).

Herein, glass compositions are expressed in terms of wt. % amounts of particular components included therein on an oxide bases unless otherwise indicated. Any component having more than one oxidation state may be present in a glass composition in any oxidation state. However, concentrations of such component are expressed in terms of the oxide in which such component is at its lowest oxidation state unless otherwise indicated.

Oral diseases pose a major health burden worldwide, causing pain, discomfort, disfigurement, and even death. The dissolution of apatite crystals and the net loss of calcium, phosphate, and other ions from the tooth (i.e., demineralization) leads to dental caries formation. Caries can be managed non-invasively through a remineralization process, in which calcium and phosphate ions are supplied from an external source to the tooth to promote crystal deposition into voids in demineralized enamel. Calcium phosphate phases in both crystalline form (e.g., brushite, β-tricalcium phosphate, octocalcium phosphate, hydroxyapatite, fluorapatite and enamel apatite) and amorphous form have been used in remineralization processes. Use of amorphous calcium phosphate (e.g., bioactive glass) in remineralization processes has shown promising results. There is a strong desire to develop new glass compositions that promote the remineralization process to prevent or repair tooth caries.

Fluorides act as caries preventive agents to inhibit demineralization and promote remineralization of dental hard tissues. Interactions of fluoride with dental hard tissues can produce a more stable acid-resistant mineral phase. The reaction products after fluoridation have include calcium fluoride (CaF₂), calcium hydroxide (Ca(OH)₂), and fluorapatite (FAP, Ca₅(PO₄)₃F). Fluoride-containing compounds can provide acid resistance and serve as fluoride reservoirs. In particular, the solubility of fluorapatite (Kₛₚ=3.19×10⁻⁶¹) is much lower than hydroxyapatite (Kₛₚ=7.36×10⁻⁶⁰). Topical fluoride delivery for caries prevention includes fluoride rinses, fluoridated toothpastes, topical fluoride gels and solutions, and fluoride varnishes. Fluoridated toothpastes generally are cost-effective and widely used products among the topical methods. In some toothpastes, NaF, SnF₂, and/or Na₂PO₃F may be formulated as a fluoride source in toothpaste or other oral compositions. However, certain disadvantages exist with such fluoride sources are used in combination with a bioactive glass, and therefore in some aspects such fluoride sources are not contained in certain compositions disclosed herein.

Bioactive glasses are a group of glass and/or glass ceramic materials that have shown biocompatibility or bioactivity, which has allowed them to be incorporated into human or animal physiology. In some aspects, disclosed is a glass composition, e.g., a bioactive glass composition, comprising SiO₂, CaO, and P₂O₅. In some aspects, the glass composition further comprises ZrO₂ and/or F. In some aspects, the glass composition forms a bioactive crystalline phase when subjected to an aqueous composition. In some aspects, the formation of the bioactive crystalline phase takes place within 15 days, within 14 days, within 13 days, within 12 days, within 11 days, within 10 days, within 9 days, within 9 days, 7 days, within 6 days, within 5 days, within 4 days, within 3 days, within 2 days, or within 1 day of subjecting to the aqueous composition. In some aspects, the aqueous composition is or comprises artificial saliva. In some aspects, the aqueous composition is or comprises real saliva (e.g., from an organism, such as a mammal, a human, and the like). In some aspects, the bioactive crystalline phase formation takes place in vivo, e.g., in real saliva in an organism, e.g., when brushing teeth of the organism or otherwise subjecting the teeth of the organism to the glass composition or a matrix thereof.

In some aspects, the aqueous composition and/or artificial saliva comprises phosphate, calcium, fluoride, sodium, potassium, magnesium, a buffer, or any combination thereof. In some aspects, the aqueous composition and/or artificial saliva comprises potassium chloride, ammonium chloride, potassium dihydrogen phosphate, magnesium chloride hexahydrate, calcium chloride, HEPES buffer, sodium azide, sodium hydroxide, a combination thereof, or any combination thereof. In some aspects, simulated body fluid may include a salt solution comprising NaCl, NaHCO₃, KCl, K₂HPO₄, MgCl₂-6H₂O, CaCl₂), NaSO₄, (HOCH₂)₃CNH₂ in nano-pure water, with pH adjusted with acid, such as HCl. In some examples, the simulated body fluid comprises artificial saliva. In some aspects, an artificial saliva composition can comprise one or more of the components set forth below in Tables 1A and 1B.

TABLE 1A

| Component | Wt. % Range |
|---|---|
| Potassium Chloride | 0.08-0.17 |
| Ammonium Chloride | 0.01-0.04 |
| Potassium Dihydrogen Phosphate | 0.03-0.08 |
| Magnesium Chloride Hexahydrate | 0.001-0.007 |
| Calcium Chloride | 0.007-0.023 |
| HEPES Buffer | 0.38-0.55 |
| Sodium Azide | 0.01-0.04 |
| Sodium Hydroxide | 0.001-0.025 |
| Water | Balance |

TABLE 1B

| Component | Wt. % Specific Example |
|---|---|
| Potassium Chloride | 0.119 |
| Ammonium Chloride | 0.024 |
| Potassium Dihydrogen Phosphate | 0.054 |
| Magnesium Chloride Hexahydrate | 0.004 |
| Calcium Chloride | 0.011 |
| HEPES Buffer | 0.477 |
| Sodium Azide | 0.02 |
| Sodium Hydroxide | 0.012 |
| Water | Balance |

Without wishing to be bound by theory, it is believed that one or more components of the artificial saliva, which has chemical composition similarities to real saliva, facilitates formation of a bioactive crystalline phase. For example, in some aspects, it is believed that one or more of phosphate, calcium, fluoride, sodium, potassium, magnesium, a buffer, or any combination thereof, but particularly the phosphate, calcium, fluoride, or a combination thereof, facilitates formation of a bioactive crystalline phase.

In some aspects, the glass compositions herein form a bioactive crystalline phase when subjected to the artificial saliva of Table 1A or Table 1B. In some aspects, the glass compositions herein form a bioactive crystalline phase when subjected to an aqueous composition and/or artificial saliva, wherein the aqueous composition and/or artificial saliva comprises one or more of potassium chloride, ammonium chloride, potassium dihydrogen phosphate, magnesium chloride hexahydrate, calcium chloride, HEPES buffer, sodium azide, sodium hydroxide, NaCl, NaHCO₃, NaSO₄, (HOCH₂)₃₃CNH₂ in nano-pure water, or any combination thereof. For example, the artificial saliva may comprise phosphate, calcium, fluoride, sodium, potassium, magnesium, a buffer, or any combination thereof.

In some aspects, the glass composition comprises a combination of SiO₂, CaO, and P₂O₅. In some aspects, the glass composition further comprises ZrO₂. In some aspects, the glass composition further comprises F. In some aspects, the glass composition further also comprises MgO, SrO, ZnO, or any combination thereof. In some aspects, the glass composition further comprises Li₂O, Na₂O, K₂O, or any combination thereof. In some aspects, the amount of Li₂O+Na₂O+K₂O is less than or equal to 10 wt. %. In some aspects, the glass composition further comprises Al₂O₃, B₂O₃, or a combination thereof.

In some aspects, the glass composition comprises SiO₂, CaO, P₂O₅, ZrO₂, and F. In some aspects, when subjected to artificial saliva of Table 1A or Table 1B, such a glass composition forms a bioactive crystalline phase.

In some aspects, the glass composition comprises SiO₂ in an amount of 15-50 wt. %. In some aspects, the glass composition comprises CaO in an amount of 25-60 wt. %. In some aspects, the glass composition comprises P₂O₅ in an amount of 3-30 wt. %. In some aspects, the glass composition comprises ZrO₂ in an amount of 0.5-15 wt. %. In some aspects, the glass composition comprises F in an amount of >0-5 wt. %.

In some aspects, the glass composition comprises:
15-50 wt. % SiO₂,
25-60 wt. % CaO,
3-30 wt. % P₂O₅,
0.5-15 wt. % ZrO₂, and
>0-5 wt. % F,
wherein, when subjected to artificial saliva of Table 1A or Table 1B, the glass composition forms a bioactive crystalline phase.

In some aspects, the glass compositions comprise:
25-45 wt. % SiO₂,
30-50 wt. % CaO,
5-25 wt. % P₂O₅,
2-10 wt. % ZrO₂, and
0.01-3 wt. % F,
wherein, when subjected to artificial saliva of Table 1A or Table 1B, the glass composition forms a bioactive crystalline phase.

The bioactivity is influenced by the composition of the glass.

Silicon dioxide (SiO₂) may serve as the primary glass-forming oxide component of the glass compositions disclosed herein. SiO₂ may be included to provide high temperature stability and chemical durability. However, if too much SiO₂ is included, such as a glass containing pure SiO₂, the melting temperature is too high to be readily workable (e.g., greater than 200 poise temperature). In addition, glass compositions including too much SiO₂ may suffer from decreased bioactivity.

In some aspects, the glass compositions comprise SiO₂ in an amount of at least 15 wt. %. In some aspects, the glass compositions comprise SiO₂ in an amount of 50 wt. % or less. In some aspects, the glass compositions comprise SiO₂ in an amount (wt. %) of 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45, or 45-50.

Phosphorus pentoxide (P₂O₅) may serve as a network former in glass compositions. Furthermore, the liberation of phosphate ions to the surface of bioactive glasses contributes to the formation of apatite. Apatite is an inorganic mineral in bone and teeth, and formation of apatite in a simulated body fluid, such as artificial saliva, is one criterion for a material to be bioactive, according to ASTM F1538-03 (2017), hereby incorporated by reference in its entirety for all purposes. The inclusion of phosphate ions in the bioactive glass increases apatite formation rate and the binding capacity of the hard tissues (e.g., bone, tooth, etc.). In addition, P₂O₅ can increase the viscosity of the glass, which in turn expands the range of operating temperatures, and is therefore an advantage to the manufacture and formation of the glass.

In some aspects, the glass compositions comprise P₂O₅ in an amount of at least 3 wt. %. In some aspects, the glass compositions comprise P₂O₅ in an amount of 30 wt. % or less. In some aspects, the glass compositions comprise P₂O₅ in an amount (wt. %) of 3-30, 3-25, 3-20, 3-15, 3-10, 3-5, 5-30, 5-25, 5-20, 5-15, 5-10, 10-30, 10-25, 10-20, 10-15, 15-30, 15-25, 15-20, 20-30, 20-25, or 25-30.

Zirconium dioxide (ZrO₂) may also be present and serves to function as a network former or intermediate in precursor glasses, as well as a key oxide for improving glass thermal stability by significantly reducing glass devitrification during forming and lowering liquidus temperature. In aspects, ZrO₂ may play a similar role as alumina (Al₂O₃) in the composition.

In some aspects, the glass compositions comprise ZrO₂ in an amount of at least 0.5 wt. %. In some aspects, the glass compositions comprise ZrO₂ in an amount of 15 wt. % or less. In some aspects, the glass compositions comprise ZrO₂ in an amount (wt. %) of 0.5-15, 0.5-14, 0.5-12, 0.5-10, 0.5-8, 0.5-6, 0.5-4, 0.5-2, 0.5-1, 1-15, 1-14, 1-12, 1-10, 1-8, 1-6, 1-4, 1-2, 2-15, 2-14, 2-12, 2-10, 2-8, 2-6, 2-4, 4-15, 4-14, 4-12, 4-10, 4-8, 4-6, 6-15, 6-14, 6-12, 6-10, 6-8, 8-15, 8-14, 8-12, 8-10, 10-15, 10-14, 10-12, 12-15, 12-14, or 14-15.

In some aspects, the glass compositions comprise fluoride (F), which facilitates remineralization of enamel. Such remineralization can form bioactive crystalline phases, such as hydroxyapatite, fluorapatite, carbonated apatite, or any combination thereof. In some aspects, at least some of such bioactive crystalline phases are resistant to acid corrosion, such as fluorapatite. F can combine with CaO and P₂O₅ to form fluorapatite to improve the bioactivity of the glass compositions. Fluorapatite is an inorganic mineral in dental enamel. The ability to form fluorapatite can help regeneration of the enamel that has become demineralized, for example, due to tooth caries (e.g., cavities). In some aspects, formation of apatite ensure the high bioactivity of the compositions disclosed herein.

In some aspects, the F in the glass compositions is derived from calcium fluoride, magnesium fluoride, sodium fluoride, potassium fluoride, stannous fluoride, sodium monofluorophosphate, sodium difluorophosphate, or any combination thereof.

In some aspects, the glass compositions comprise F⁻ in an amount of 0 wt. % or greater than 0 wt. %. In some aspects, the glass compositions comprise F⁻ in an amount of 5 wt. % or less. In some aspects, the glass compositions comprise F in an amount (wt. %) of 0-5, 0-4.5, 0-4, 0-3.5, 0-3, 0-2.5, 0-2, 0-1.5, 0-1, 0-0.5, >0-5, >0-4.5, >0-4, >0-3.5, >0-3, >0-2.5, >0-2, >0-1.5, >0-1, >0-0.5, >0-0.4, >0-0.3, >0-0.2, >0-0.1, >0-0.01, 0.01-5, 0.01-4.5, 0.01-4, 0.01-3.5, 0.01-3, 0.01-2.5, 0.01-2, 0.01-1.5, 0.01-1, 0.01-0.5, 0.01-0.4, 0.01-0.3, 0.01-0.2, 0.01-0.1, 0.1-5, 0.1-4.5, 0.1-4, 0.1-3.5, 0.1-3, 0.1-2.5, 0.1-2, 0.1-1.5, 0.1-1, 0.1-0.5, 0.1-0.4, 0.1-0.3, 0.1-0.2, 0.2-5, 0.2-4.5, 0.2-4, 0.2-3.5, 0.2-3, 0.2-2.5, 0.2-2, 0.2-1.5, 0.2-1, 0.2-0.5, 0.2-0.4, 0.2-0.3, 0.3-5, 0.3-4.5, 0.3-4, 0.3-3.5, 0.3-3, 0.3-2.5, 0.3-2, 0.3-1.5, 0.3-1, 0.3-1.5, 0.3-0.4, 0.4-5, 0.4-4.5, 0.4-4, 0.4-3.5, 0.4-3, 0.4-2.5, 0.4-2, 0.4-1.5, 0.4-1, 0.4-0.5, 0.5-5, 0.5-4.5, 0.5-4, 0.5-3.5, 0.5-3, 0.5-2.5, 0.5-2, 0.5-1.5, 0.5-1, 1-5, 1-4.5, 1-4, 1-3.5, 1-3, 1-2.5, 1-2, 1-1.5, 1.5-5, 1.5-4.5, 1.5-4, 1.5-3.5, 1.5-3, 1.5-2.5, 1.5-2, 2-5, 2-4.5, 2-4, 2-3.5, 2-3, 2-2.5, 2.5-5, 2.5-4.5, 2.5-4, 2.5-3.5, 2.5-3, 3-5, 3-4.5, 3-4, 3-3.5, 3.5-5, 3.5-4.5, 3.5-4, 4-5, 4-4.5, or 4.5-5.

Divalent cation oxides (such as alkaline earth oxides and ZnO) improve the melting behavior, chemical durability, and bioactivity of the glass. In addition, alkaline earth oxides may improve other desirable properties in the materials, including influencing the Young's modulus and the coefficient of thermal expansion. In examples, the glass compositions comprise from 30-70 wt. % MO, wherein MO is the sum of MgO, CaO, SrO, BeO, and BaO. In some aspects, the glass compositions comprise MO in an amount (wt. %) of 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70.

In some aspects, the glass compositions comprise CaO in an amount of at least 25 wt. %. In some aspects, the glass compositions comprise CaO in an amount of 60 wt. % or less. In some aspects, the glass compositions comprise CaO in an amount (wt. %) of 25-60, 25-55, 25-50, 25-45, 25-40, 25-35, 25-30, 30-60, 30-55, 30-50, 30-45, 30-40, 30-35, 35-60, 35-55, 35-50, 35-45, 35-40, 40-60, 40-55, 40-50, 40-45, 45-60, 45-55, 45-50, 50-60, 50-55, or 55-60.

In some aspects, the glass compositions comprise MgO. In some aspects, the inclusion of MgO can improve liquidus of the precursor glass to avoid devitrification during forming. In some aspects, the glass compositions comprise MgO in an amount of 0 wt. % or greater than 0 wt. %. In some aspects, the glass compositions comprise MgO in an amount of 25 wt. % or less. In some aspects, the glass compositions comprise MgO in an amount (wt. %) of 0-25, 0-20, 0-15, 0-10, 0-5, >0-25, >0-20, >0-15, >0-10, >0-5, 5-25, 5-20, 5-15, 5-10, 10-25, 10-20, 10-15, 15-25, 15-20, or 20-25.

In some aspects, the glass compositions comprise SrO. In some aspects, the inclusion of SrO can improve liquidus of the precursor glass to avoid devitrification during forming. Moreover, in some aspects, SrO may also enter the structure of apatite to improve bioactivity. In some aspects, the glass compositions comprise SrO in an amount of 0 wt. % or greater than 0 wt. %. In some aspects, the glass compositions comprise SrO in an amount of 10 wt. % or less. In some aspects, the glass compositions comprise SrO in an amount (wt. %) of 0-10, 0-9, 0-8, 0-7, 0-6, 0-5, 0-4, 0-3, 0-2, 0-1, 0-0.5, 0-0.1, >0-10, >0-9, >0-8, >0-7, >0-6, >0-5, >0-4, >0-3, >0-2, >0-1, >0-0.5, >0-0.1, 0.1-10, 0.1-9, 0.1-8, 0.1-7, 0.1-6, 0.1-5, 0.1-4, 0.1-3, 0.1-2, 0.1-1, 0.1-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10.

In some aspects, the glass compositions comprise ZnO. In some aspects, the inclusion of ZnO can improve liquidus of the precursor glass to avoid devitrification during forming. Moreover, in some aspects, ZnO may also enter the structure of apatite to improve bioactivity. In some aspects, the glass compositions comprise ZnO in an amount of 0 wt. % or greater than 0 wt. %. In some aspects, the glass compositions comprise ZnO in an amount of 10 wt. % or less. In some aspects, the glass compositions comprise ZnO in an amount (wt. %) of 0-10, 0-8, 0-6, 0-4, 0-2, >0-10, >0-8, >0-6, >0-4, >0-2, 2-10, 2-8, 2-6, 2-4, 4-10, 4-8, 4-6, 6-10, 6-8, or 8-10.

In some aspects, CaO is found to be able to react with P₂O₅ to form apatite when immersed in a simulated body fluid (SBF), such as artificial saliva, or in vivo or in real saliva. The release of Ca²⁺ ions from the surface of the glass contributes to the formation of a layer rich in calcium phosphate. Thus, the combination of P₂O₅ and CaO may provide advantageous compositions for bioactive glasses.

In some aspects, the glass compositions comprise the sum P₂O₅+CaO in an amount of 40 wt. % or more. In some aspects, the glass compositions comprise the sum P₂O₅+CaO in an amount of 70 wt. % or less. In some aspects, the glass compositions comprise the sum P₂O₅+CaO in an amount (wt. %) of 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70.

In some aspects, the glass compositions comprise the sum MgO+CaO in an amount of at least 30 wt. %. In some aspects, the glass compositions comprise the sum MgO+CaO in an amount of 70 wt. % or less. In some aspects, the glass compositions comprise the sum MgO+CaO in an amount (wt. %) of 30-70, 30-65, 30-60, 30-55, 30-50, 30-45, 30-40, 30-35, 35-70, 35-65, 35-60, 35-55, 35-50, 35-45, 35-40, 40-70, 40-65, 40-60, 40-55, 40-50, 40-45, 45-70, 45-65, 45-60, 45-55, 45-50, 50-70, 50-65, 50-60, 50-55, 55-70, 55-65, 55-60, 60-70, 60-65, or 65-70.

In some aspects, the glass compositions comprise the sum F+P₂O₅ in an amount of at least 5 wt. %. In some aspects, the glass compositions comprise the sum F+P₂O₅ in an amount of 30 wt. % or less. In some aspects, the glass compositions comprise the sum F+P₂O₅ in an amount (wt. %) of 5-30, 5-25, 5-20, 5-15, 5-10, 10-30, 10-25, 10-20, 10-15, 15-30, 15-25, 15-20, 20-30, 20-25, or 25-30.

In some aspects, the glass compositions comprise B₂O₃ in an amount of 0 wt. % or greater than 0 wt. %. In some aspects, the glass compositions comprise B₂O₃ in an amount of 5 wt. % or less. In some aspects, the glass compositions comprise B₂O₃ in an amount (wt. %) of 0-5, 0-4.5, 0-4, 0-3.5, 0-3, 0-2.5, 0-2, 0-1.5, 0-1, 0-0.5, 0-0.4, 0-0.3, 0-0.2, 0-0.1, 0-0.05, >0-5, >0-4.5, >0-4, >0-3.5, >0-3, >0-2.5, >0-2, >0-1.5, >0-1, >0-0.5, >0-0.4, >0-0.3, >0-0.2, >0-0.1, >0-0.05, 0.05-5, 0.05-4.5, 0.05-4, 0.05-3.5, 0.05-3, 0.05-2.5, 0.05-2, 0.05-1.5, 0.05-1, 0.05-0.5, 0.05-0.4, 0.05-0.3, 0.05-0.2, 0.05-0.1, 0.1-5, 0.1-4.5, 0.1-4, 0.1-3.5, 0.1-3, 0.1-2.5, 0.1-2, 0.1-1.5, 0.1-1, 0.1-0.5, 0.1-0.4, 0.1-0.3, 0.1-0.2, 0.2-5, 0.2-4.5, 0.2-4, 0.2-3.5, 0.2-3, 0.2-2.5, 0.2-2, 0.2-1.5, 0.2-1, 0.2-0.5, 0.2-0.4, 0.2-0.3, 0.3-5, 0.3-4.5, 0.3-4, 0.3-3.5, 0.3-3, 0.3-2.5, 0.3-2, 0.3-1.5, 0.3-1, 0.3-0.5, 0.3-0.4, 0.4-5, 0.4-4.5, 0.4-4, 0.4-3.5, 0.4-3, 0.4-2.5, 0.4-2, 0.4-1.5, 0.4-1, 0.4-0.5, 0.5-5, 0.5-4.5, 0.5-4, 0.5-3.5, 0.5-3, 0.5-2.5, 0.5-2, 0.5-1.5, 0.5-1, 1-5, 1-4.5, 1-4, 1-3.5, 1-3, 1-2.5, 1-2, 1-1.5, 1.5-5, 1.5-4.5, 1.5-4, 1.5-3.5, 1.5-3, 1.5-2.5, 1.5-2, 2-5, 2-4.5, 2-4, 2-3.5, 2-3, 2-2.5, 2.5-5, 2.5-4.5, 2.5-4, 2.5-3.5, 2.5-3, 3-5, 3-4.5, 3-4, 3-3.5, 3.5-5, 3.5-4.5, 3.5-4, 4-5, 4-4.5, or 4.5-5.

In some aspects, the glass compositions can comprise Al₂O₃. In some aspects, Al₂O₃ may influence (e.g., stabilize) the network structure of the glass, improve mechanical properties, improve chemical durability, or any combination thereof. In some aspects, Al₂O₃ may lower liquidus temperature, lower coefficient of thermal expansion, enhance the strain point, or any combination thereof. In some aspects, in addition to its role as a network former, Al₂O₃ (and ZrO₂) may help improve the chemical durability and mechanical properties in silicate glass while having no toxicity concerns. In some aspects, too high a content of Al₂O₃ or ZrO₂ (e.g., >20 wt. %) generally increases the viscosity of the melt and decreases bioactivity.

In some aspects, the glass compositions comprise Al₂O₃ in an amount of 0 wt. % or greater than 0 wt. %. In some aspects, the glass compositions comprise Al₂O₃ in an amount of 5 wt. % or less. In some aspects, the glass compositions comprise Al₂O₃ in an amount (wt. %) of 0-5, 0-4.5, 0-4, 0-3.5, 0-3, 0-2.5, 0-2, 0-1.5, 0-1, 0-0.5, 0-0.4, 0-0.3, 0-0.2, 0-0.1, 0-0.05, >0-5, >0-4.5, >0-4, >0-3.5, >0-3, >0-2.5, >0-2, >0-1.5, >0-1, >0-0.5, >0-0.4, >0-0.3, >0-0.2, >0-0.1, >0-0.05, 0.05-5, 0.05-4.5, 0.05-4, 0.05-3.5, 0.05-3, 0.05-2.5, 0.05-2, 0.05-1.5, 0.05-1, 0.05-0.5, 0.05-0.4, 0.05-0.3, 0.05-0.2, 0.05-0.1, 0.1-5, 0.1-4.5, 0.1-4, 0.1-3.5, 0.1-3, 0.1-2.5, 0.1-2, 0.1-1.5, 0.1-1, 0.1-0.5, 0.1-0.4, 0.1-0.3, 0.1-0.2, 0.2-5, 0.2-4.5, 0.2-4, 0.2-3.5, 0.2-3, 0.2-2.5, 0.2-2, 0.2-1.5, 0.2-1, 0.2-0.5, 0.2-0.4, 0.2-0.3, 0.3-5, 0.3-4.5, 0.3-4, 0.3-3.5, 0.3-3, 0.3-2.5, 0.3-2, 0.3-1.5, 0.3-1, 0.3-0.5, 0.3-0.4, 0.4-5, 0.4-4.5, 0.4-4, 0.4-3.5, 0.4-3, 0.4-2.5, 0.4-2, 0.4-1.5, 0.4-1, 0.4-0.5, 0.5-5, 0.5-4.5, 0.5-4, 0.5-3.5, 0.5-3, 0.5-2.5, 0.5-2, 0.5-1.5, 0.5-1, 1-5, 1-4.5, 1-4, 1-3.5, 1-3, 1-2.5, 1-2, 1-1.5, 1.5-5, 1.5-4.5, 1.5-4, 1.5-3.5, 1.5-3, 1.5-2.5, 1.5-2, 2-5, 2-4.5, 2-4, 2-3.5, 2-3, 2-2.5, 2.5-5, 2.5-4.5, 2.5-4, 2.5-3.5, 2.5-3, 3-5, 3-4.5, 3-4, 3-3.5, 3.5-5, 3.5-4.5, 3.5-4, 4-5, 4-4.5, or 4.5-5.

In some aspects, alkali oxides (Na₂O, K₂O, Li₂O, Rb₂O, or Cs₂O) serve as aids in achieving low melting temperature and low liquidus temperatures, which can aid glass melting processes. In addition, in some aspects, the addition of alkali oxides can improve bioactivity. However, in some aspects, if the amount of alkali oxides is too high, the glass compositions have reduced chemical durability; in other words, in some aspects, keeping the amount of alkali oxides to a certain lower range achieves high chemical durability of the glass compositions. In some aspects, the glass compositions do not contain any alkali oxides (e.g., do not contain any added alkali oxides). In some aspects, the glass compositions are substantially free of alkali oxides (e.g., contain less than 0.1 wt. % alkali oxides).

In some aspects, the glass compositions comprise Li₂O in an amount of 0 wt. % or greater than 0 wt. %. In some aspects, the glass compositions comprise Li₂O in an amount of 10 wt. % or less. In some aspects, the glass compositions comprise Li₂O in an amount (wt. %) of 0-10, 0-9, 0-8, 0-7, 0-6, 0-5, 0-4, 0-3, 0-2, 0-1, 0-0.5, 0-0.1, >0-10, >0-9, >0-8, >0-7, >0-6, >0-5, >0-4, >0-3, >0-2, >0-1, >0-0.5, >0-0.1, 0.1-10, 0.1-9, 0.1-8, 0.1-7, 0.1-6, 0.1-5, 0.1-4, 0.1-3, 0.1-2, 0.1-1, 0.1-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10.

In some aspects, the glass compositions comprise Na₂O in an amount of 0 wt. % or greater than 0 wt. %. In some aspects, the glass compositions comprise Na₂O in an amount of 10 wt. % or less. In some aspects, the glass compositions comprise Na₂O in an amount (wt. %) of 0-10, 0-9, 0-8, 0-7, 0-6, 0-5, 0-4, 0-3, 0-2, 0-1, 0-0.5, 0-0.1, >0-10, >0-9, >0-8, >0-7, >0-6, >0-5, >0-4, >0-3, >0-2, >0-1, >0-0.5, >0-0.1, 0.1-10, 0.1-9, 0.1-8, 0.1-7, 0.1-6, 0.1-5, 0.1-4, 0.1-3, 0.1-2, 0.1-1, 0.1-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10.

In some aspects, the glass compositions comprise K₂O in an amount of 0 wt. % or greater than 0 wt. %. In some aspects, the glass compositions comprise K₂O in an amount of 10 wt. % or less. In some aspects, the glass compositions comprise K₂O in an amount (wt, %) of 0-10, 0-9, 0-8, 0-7, 0-6, 0-5, 0-4, 0-3, 0-2, 0-1, 0-0.5, 0-0.1, >0-10, >0-9, >0-8, >0-7, >0-6, >0-5, >0-4, >0-3, >0-2, >0-1, >0-0.5, >0-0.1, 0.1-10, 0.1-9, 0.1-8, 0.1-7, 0.1-6, 0.1-5, 0.1-4, 0.1-3, 0.1-2, 0.1-1, 0.1-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10.

In some aspects, the glass compositions comprise the sum Li₂O+Na₂O+K₂O in an amount of 0 wt. % or greater than 0 wt. %. In some aspects, the glass compositions comprise the sum Li₂O+Na₂O+K₂O in an amount of 10 wt. % or less. In some aspects, the glass compositions comprise the sum Li₂O+Na₂O+K₂O in an amount (wt. %) of 0-10, 0-9, 0-8, 0-7, 0-6, 0-5, 0-4, 0-3, 0-2, 0-1, 0-0.5, 0-0.1, >0-10, >0-9, >0-8, >0-7, >0-6, >0-5, >0-4, >0-3, >0-2, >0-1, >0-0.5, >0-0.1, 0.1-10, 0.1-9, 0.1-8, 0.1-7, 0.1-6, 0.1-5, 0.1-4, 0.1-3, 0.1-2, 0.1-1, 0.1-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10. In some aspects, the glass compositions comprise the sum Na₂O+K₂O+Li₂O+Rb₂O+Cs₂O in any of the amounts set forth in this paragraph.

In some aspects, additional components can be incorporated into the glass compositions to provide additional benefits or may be incorporated as contaminants typically found in commercially-prepared glass. For example, additional components can be added as coloring or fining agents (e.g., to facilitate removal of gaseous inclusions from melted batch materials used to produce the glass compositions) and/or for other purposes. In some aspects, the glass compositions may comprise one or more compounds useful as ultraviolet radiation absorbers. In some aspects, the glass compositions can comprise 3 wt. % or less ZnO, TiO₂, CeO, MnO, Nb₂O₅, MoO₃, Ta₂O₅, WO₃, SnO₂, Fe₂O₃, As₂O₃, Sb₂O₃, Cl, Br, or any combination thereof. In some aspects, the glass compositions can comprise from 0 to about 3 wt. %, 0 to about 2 wt. %, 0 to about 1 wt. %, 0 to 0.5 wt. %, 0 to 0.1 wt. %, 0 to 0.05 wt. %, or 0 to 0.01 wt. % ZnO. TiO₂, CeO, MnO, Nb₂O₅, MoO₃, Ta₂O₅, WO₃, SnO₂, Fe₂O₃, As₂O₃, Sb₂O₃, Cl, Br, or any combination thereof. In some aspects, the glass compositions can also include various contaminants associated with batch materials and/or introduced into the glass by the melting, fining, and/or forming equipment used to produce the glass. For example, in some aspects, the glass compositions can comprise from 0 to about 3 wt. %, 0 to about 2 wt. %, 0 to about 1 wt. %, 0 to about 0.5 wt. %, 0 to about 0.1 wt. %, 0 to about 0.05 wt. %, or 0 to about 0.01 wt. % SnO₂ or Fe₂O₃, or a combination thereof.

In some aspects, the glass composition is substantially free of, or does not contain, a crystalline phase. For example, in some aspects, the glass composition has a glassy phase and no crystalline phase. In some aspects, the glass composition is a glass (e.g., a glass article). In some aspects, the glass composition is a glass-ceramic (e.g., a glass-ceramic article). In some aspects, the glass composition contains a crystalline phase.

In some aspects, the glass composition is in a form of particulates, microbeads, fibers, or a combination thereof. In some aspects, the particulates are a fine powder. Methods for preparing particulates, microbeads, and/or fibers from glass compositions is known in the art. In some aspects, the particulates, microbeads, and/or fibers have an average particle size of at least 1 micron. In some aspects, the particulates and/or microbeads have an average particle size of 50 microns or less. In some aspects, the particulates and/or microbeads have an average particle size (microns) of 1-50, 1-45, 1-40, 1-35, 1-30, 1-25, 1-20, 1-15, 1-10, 1-5, 5-50, 5-45, 5-40, 5-35, 5-30, 5-25, 5-20, 5-15, 5-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45, or 45-50. Average particle size, as used herein, means the average size as measured by dynamic light scattering using commercially available equipment.

In some aspects, when subjected to an aqueous composition (e.g., artificial saliva or real saliva), as described elsewhere herein, the glass composition forms a bioactive crystalline phase. In some aspects, the formation of the bioactive crystalline phase takes place within a certain timeframe, such as within 1, 2, 3, 4, 5, 6, or 7 days, as described elsewhere herein. In some aspects, the bioactive crystalline phase comprises apatite. In some aspects, the apatite comprises hydroxyapatite, fluorapatite, carbonated apatite, or any combination thereof.

Aspects are related to compositions or matrices containing embodied bioactive glass compositions and the methods of using the matrices to treat medical conditions. The matrices can be a toothpaste, mouthwash, rinse, spray, ointment, salve, cream, bandage, polymer film, oral formulation, pill, capsule, transdermal formulation, and the like. The bioactive glass compositions disclosed can be physically or chemically attached to matrices or other matrix components, or simply mixed in. As noted above, the bioactive glass can be in any form that works in the application, including particles, beads, particulates, short fibers, long fibers, or woolen meshes. The methods of using the glass-containing matrices to treat a medical condition can be simply like the use of matrix as normally applied.

In some aspects, the glass compositions can be made via traditional methods. For example, in some aspects, precursor glasses can be formed by thoroughly mixing the requisite batch materials (for example, using a Turbula® mixer) in order to secure a homogeneous melt, and subsequently placing into silica and/or platinum crucibles. The crucibles can be placed into a furnace and the glass batch melted and maintained at temperatures ranging from 1100° C. to 1400° C. for times ranging from about 6 hours to 24 hours. The melts can thereafter be poured into steel molds to yield glass slabs. Subsequently, those slabs optionally can be transferred immediately to an annealer operating at about 400° C. to 700° C., where the glass is held at temperature for about 0.5 hour to 3 hours and subsequently cooled overnight. In another non-limiting example, precursor glasses are prepared by dry blending the appropriate oxides and mineral sources for a time sufficient to thoroughly mix the ingredients. The glasses are melted in platinum crucibles at temperatures ranging from about 1100° C. to 1400° C. and held at temperature for about 6 hours to 16 hours. The resulting glass melts are then poured onto a steel table to cool. The precursor glasses optionally are then annealed at appropriate temperatures.

In some aspects, the resulting glass compositions can be ground into fine particles in the range of 1-10 microns (μm) by air jet milling. The particle size can be varied in the range of 1-100 μm using attrition milling or ball milling of glass frits. Furthermore, these glasses can be processed into short fibers, beads, sheets or three-dimensional scaffolds using different methods. Short fibers are made by melt spinning or electric spinning; beads can be produced by flowing glass particles through a hot vertical furnace or a flame torch; sheets can be manufactured using thin rolling, float or fusion-draw processes; and scaffolds can be produced using rapid prototyping, polymer foam replication and particle sintering. Glasses of desired forms can be used to support cell growth, soft and hard tissue regeneration, stimulation of gene expression or angiogenesis.

Continuous fibers can be easily drawn from the disclosed compositions using processes known in the art. For example, fibers can be formed using a directly heated (electricity passing directly through) platinum bushing. Glass cullet is loaded into the bushing, heated up until the glass can melt. Temperatures are set to achieve a desired glass viscosity (usually <1000 poise) allowing a drip to form on the orifice in the bushing (Bushing size is selected to create a restriction that influences possible fiber diameter ranges). The drip is pulled by hand to begin forming a fiber. Once a fiber is established it is connected to a rotating pulling/collection drum to continue the pulling process at a consistent speed. Using the drum speed (or revolutions per minute RPM) and glass viscosity the fiber diameter can be manipulated—in general the faster the pull speed, the smaller the fiber diameter. Glass fibers with diameters in the range of 1-100 μm can be drawn continuously from a glass melt. Fibers can also be created using an updraw process. In this process, fibers are pulled from a glass melt surface sitting in a box furnace. By controlling the viscosity of the glass, a quartz rod is used to pull glass from the melt surface to form a fiber. The fiber can be continuously pulled upward to increase the fiber length. The velocity that the rod is pulled up determines the fiber thickness along with the viscosity of the glass.

Therefore, in some aspects, a method for making the glass compositions disclosed herein is provided, the method comprising:
combining each component of the glass composition to form a mixture,
heating the mixture to a temperature of 1500° C. or less to form a melted mixture, and
cooling the melted mixture to form a cooled mixture.

In some aspects, the method further comprising forming the cooled mixture into particulates, microbeads, fibers, or a combination thereof.

In some aspects, disclosed is a matrix comprising the glass compositions. In some aspects, the matrix comprises a glass composition, wherein the glass composition is attached to the matrix or mixed therein. In some aspects, the matrix includes at least one of: a toothpaste, mouthwash, gel, varnish, rinse, spray, ointment, salve, cream, bandage, polymer film, oral formulation, pill, capsule, or transdermal formulation.

In some aspects, the matrix is substantially free of, or does not include, a fluoride ion source that is derived from the glass composition (e.g., a fluoride ion source that is added to the matrix as a component separate from the glass composition and which fluoride ion source is not derived from the glass composition). In this regard, in some aspects, the matrix is substantially free of, or does not contain, calcium fluoride, magnesium fluoride, sodium fluoride, potassium fluoride, stannous fluoride, sodium monofluorophosphate, sodium difluorophosphate, or any combination thereof. In some aspects, the matrix is substantially free of, or does not contain, a source of fluoride ions that is added to the matrix separately from the glass composition. In some aspects, the matrix includes a fluoride ion source that is added separately to the matrix. In some aspects, the matrix comprises calcium fluoride, magnesium fluoride, sodium fluoride, potassium fluoride, stannous fluoride, sodium monofluorophosphate, sodium difluorophosphate, or any combination thereof. In some aspects, the calcium fluoride, magnesium fluoride, sodium fluoride, potassium fluoride, stannous fluoride, sodium monofluorophosphate, sodium difluorophosphate, or any combination thereof are added to the matrix separately from the glass composition.

In some aspects, the matrix is or comprises a dentifrice composition. In some aspects, the dentifrice composition is a toothpaste, a mouthwash, or a gel, or any other formulation that is intended for oral care. In some aspects, without wishing to be bound by theory, it is believed that high chemical durability and high bioactivity of the glass compositions makes them advantageous for use in oral care formulations (e.g., dentifrice compositions) or cosmetic formulations.

In some aspects, the dentifrice compositions can comprise any suitable components and amounts of such components. For example, in some aspects, the dentifrice compositions comprise the glass composition in combination with one or more of a solvent, a humectant, a fluoride ion source, a flavoring, a surfactant, a sweetener, a whitening agent, an abrasive, or any combination thereof. Such dentifrice compositions may be aqueous based or they may be non-aqueous based. When aqueous based, the solvent comprises water. When non-aqueous based, the solvent typically comprises glycerin. In some aspects, a suitable dentifrice composition with ranges of components is shown in Table 2.

TABLE 2

| Component | Amount (wt. %) |
|---|---|
| Glass Composition | >0-20 |
| Solvent* | 10-80 |
| Humectant | 2-50 |
| Fluoride Ion Source | 0-1 |
| Flavoring | 0-0.5 |
| Surfactant | >0-5 |
| Sweetener | 0-5 |
| Whitening Agent | 0-2 |
| Abrasive | 0-20 |
| Other | 0-10 |

*Solvent can be water, glycerin, or a combination thereof

In some aspects, the dentifrice compositions comprise the glass composition in amount of greater than 0 wt. %. In some aspects, the dentifrice compositions comprise the glass composition in amount of 20 wt. % or less. In some aspects, the dentifrice compositions comprise the glass composition in amount (wt. %) of >0-20, >0-18, >0-16, >0-14, >0-12, >0-10, >0-8, >0-6, >0-4, >0-2, >0-1, >0-0.5, >0-0.1, 0.1-20, 0.1-18, 0.1-16, 0.1-14, 0.1-12, 0.1-10, 0.1-8, 0.1-6, 0.1-4, 0.1-2, 0.1-1, 0.1-0.5, 0.5-20, 0.5-18, 0.5-16, 0.5-14, 0.5-12, 0.5-10, 0.5-8, 0.5-6, 0.5-4, 0.5-2, 0.5-1, 1-20, 1-18, 1-16, 1-14, 1-12, 1-10, 1-8, 1-6, 1-4, 1-2, 2-20, 2-18, 2-6, 2-14, 2-12, 2-10, 2-8, 2-6, 2-4, 4-20, 4-18, 4-16, 4-14, 4-12, 4-10, 4-8, 4-6, 6-20, 6-18, 6-16, 6-14, 6-12, 6-10, 6-8, 8-20, 8-18, 8-16, 8-14, 8-12, 8-10, 10-20, 10-18, 10-16, 10-14, 10-12, 12-20, 12-18, 12-16, 12-14, 14-20, 14-18, 14-16, 16-20, 16-18, or 18-20.

In some aspects, the dentifrice compositions comprise a solvent in an amount of 10 wt. % or more. In some aspects, the dentifrice compositions comprise a solvent in an amount of 80 wt. % or less. In some aspects, the dentifrice compositions comprise a solvent in an amount (wt. %) of 10-80, 10-75, 10-70, 10-65, 10-60, 10-55, 10-50, 10-45, 10-40, 10-35, 10-30, 10-25, 10-20, 10-15, 15-80, 15-75, 15-70, 15-65, 15-60, 15-55, 15-50, 15-45, 15-40, 15-35, 15-30, 15-25, 15-20, 20-80, 20-75, 20-70, 20-65, 20-60, 20-55, 20-50, 20-45, 20-40, 20-35, 20-30, 20-25, 25-80, 25-75, 25-70, 25-65, 25-60, 25-55, 25-50, 25-45, 25-40, 25-35, 25-30, 30-80, 30-75, 30-70, 30-65, 30-60, 30-55, 30-50, 30-45, 30-40, 30-35, 35-80, 35-75, 35-70, 35-65, 35-60, 35-55, 35-50, 35-45, 35-40, 40-80, 40-75, 40-70, 40-65, 40-60, 40-55, 40-50, 40-45, 45-80, 45-75, 45-70, 45-65, 45-60, 45-55, 45-50, 50-80, 50-75, 50-70, 50-65, 50-60, 50-55, 55-80, 55-75, 55-70, 55-65, 55-60, 60-80, 60-75, 60-70, 60-65, 65-80, 65-75, 65-70, 70-80, 70-75, or 75-80. As disclosed elsewhere herein, the solvent can be aqueous or non-aqueous. Generally, when aqueous, the solvent is or comprises water, and when non-aqueous, the solvent is or comprises glycerin. In some aspects, a combination of water and glycerin is employed, and the weight percent of glycerin in such a mixture can be 1-99, 5-95, 10-90, 15-85, 20-80, 25-75, 30-70, 35-65, 40-60, 45-55, 1-50, 5-45, 10-40, 15-35, 50-95, 55-90, 60-85, or 65-80.

In some aspects, the dentifrice compositions comprise one or more humectants. In some aspects, the humectants can include short-chained polyalcohols For example, short-chained polyalcohols can include glycerol, sorbitol, propylene glycol, polyethylene glycol, or any combination thereof. In some aspects, the dentifrice compositions comprise one or more humectants in an amount of 2 wt. % or more. In some aspects, the dentifrice compositions comprise one or more humectants in an amount of 50 wt. % or less. In some aspects, the dentifrice compositions comprise one or more humectants in an amount (wt. %) of 2-50, 2-45, 2-40, 2-35, 2-30, 2-25, 2-20, 2-15, 2-10, 2-5, 5-50, 5-45, 5-40, 5-35, 5-30, 5-25, 5-20, 5-15, 5-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45, or 45-50.

In some aspects, the dentifrice composition comprises one or more fluoride ion sources. In some aspects, the one or more fluoride ion sources includes calcium fluoride, magnesium fluoride, sodium fluoride, potassium fluoride, stannous fluoride, sodium monofluorophosphate, sodium difluorophosphate, or any combination thereof. In some aspects, the dentifrice compositions comprise one or more fluoride ion sources in an amount of 0 wt. % or greater than 0 wt. %. In some aspects, the dentifrice compositions comprise one or more fluoride ion sources in an amount of 1 wt. % or less. In some aspects, the dentifrice compositions comprise one or more fluoride ion sources in an amount (wt. %) of 0-1, 0-0.9, 0-0.8, 0-0.7, 0-0.6, 0-0.5, 0-0.4, 0-0.3, 0-0.2, 0-0.1, 0-0.05, >0-1, >0-0.9, >0-0.8, >0-0.7, >0-0.6, >0-0.5, >0-0.4, >0-0.3, >0-0.2, >0-0.1, >0-0.05, 0.05-1, 0.05-0.9, 0.05-0.8, 0.05-0.7, 0.05-0.6, 0.05-0.5, 0.05-0.4, 0.05-0.3, 0.05-0.2, 0.05-0.1, 0.1-1, 0.1-0.9, 0.1-0.8, 0.1-0.7, 0.1-0.6, 0.1-0.5, 0.1-0.4, 0.1-0.3, 0.1-0.2, 0.2-1, 0.2-0.9, 0.2-0.8, 0.2-0.7, 0.2-0.6, 0.2-0.5, 0.2-0.4, 0.2-0.3, 0.3-1, 0.3-0.9, 0.3-0.8, 0.3-0.7, 0.3-0.6, 0.3-0.5, 0.3-0.4, 0.4-1, 0.4-0.9, 0.4-0.8, 0.4-0.7, 0.4-0.6, 0.4-0.5, 0.5-1, 0.5-0.9, 0.5-0.8, 0.5-0.7, 0.5-0.6, 0.6-1, 0.6-0.9, 0.6-0.8, 0.6-0.7, 0.7-1, 0.7-0.9, 0.7-0.8, 0.8-1, 0.8-0.9, or 0.9-1.

In some aspects, the dentifrice compositions comprise one or more flavoring agents in an amount of 0 wt. % or greater than 0 wt. %. In some aspects, the dentifrice compositions comprise one or more flavoring agents in an amount of 0.5 wt. % or less. In some aspects, the dentifrice compositions comprise one or more flavoring agents in an amount (wt. %) of 0-0.5, 0-0.4, 0-0.3, 0-0.2, 0-0.1, 0-0.05, >0-0.5, >0-0.4, >0-0.3, >0-0.2, >0-0.1, >0-0.05, 0.05-0.5, 0.05-0.4, 0.05-0.3, 0.05-0.2, 0.05-0.1, 0.1-0.5, 0.1-0.4, 0.1-0.3, 0.1-0.2, 0.2-0.5, 0.2-0.4, 0.2-0.3, 0.3-0.5, 0.3-0.4, or 0.4-0.5.

In some aspects, the dentifrice compositions comprise one or more surfactants. In some aspects, the one or more surfactants include sodium lauryl sulfate, poloxamer (such as an ethylene oxide/propylene oxide copolymers), and any combination thereof. In some aspects, sodium lauryl sulfate exacerbates tooth sensitivity issues and therefore in some aspects sodium lauryl sulfate is not included in the dentifrice compositions. In some aspects, the dentifrice compositions comprise one or more surfactants in an amount of greater than 0 wt. %. In some aspects, the dentifrice compositions comprise one or more surfactants in an amount of 5 wt. % or less. In some aspects, the dentifrice compositions comprise one or more surfactants in an amount (wt. %) of >0-5, >0-4.5, >0-4, >0-3.5, >0-3, >0-2.5, >0-2, >0-1.5, >0-1, >0-0.5, >0-0.1, 0.1-5, 0.1-4.5, 0.1-4, 0.1-3.5, 0.1-3, 0.1-2.5, 0.1-2, 0.1-1.5, 0.1-1, 0.1-0.5, 0.5-5, 0.5-4.5, 0.5-4, 0.5-3.5, 0.5-3, 0.5-2.5, 0.5-2, 0.5-1.5, 0.5-1, 1-5, 1-4.5, 1-4, 1-3.5, 1-3, 1-2.5, 1-2, 1-1.5, 1.5-5, 1.5-4.5, 1.5-4, 1.5-3.5, 1.5-3, 1.5-2.5, 1.5-2, 2-5, 2-4.5, 2-4, 2-3.5, 2-3, 2-2.5, 2.5-5, 2.5-4.5, 2.5-4, 2.5-3.5, 2.5-3, 3-5, 3-4.5, 3-4, 3-3.5, 3.5-5, 3.5-4.5, 3.5-4, 4-5, 4-4.5, or 4.5-5.

In some aspects, the dentifrice compositions comprise one or more sweeteners in an amount of 0 wt. % or greater than 0 wt. %. In some aspects, the dentifrice compositions comprise one or more sweeteners in an amount of 5 wt. % or less. In some aspects, the dentifrice compositions comprise one or more surfactants in an amount (wt. %) of 0-5, 0-4.5, 0-4, 0-3.5, 0-3, 0-2.5, 0-2, 0-1.5, 0-1, 0-0.5, 0-0.1, >0-5, >0-4.5, >0-4, >0-3.5, >0-3, >0-2.5, >0-2, >0-1.5, >0-1, >0-0.5, >0-0.1, 0.1-5, 0.1-4.5, 0.1-4, 0.1-3.5, 0.1-3, 0.1-2.5, 0.1-2, 0.1-1.5, 0.1-1, 0.1-0.5, 0.5-5, 0.5-4.5, 0.5-4, 0.5-3.5, 0.5-3, 0.5-2.5, 0.5-2, 0.5-1.5, 0.5-1, 1-5, 1-4.5, 1-4, 1-3.5, 1-3, 1-2.5, 1-2, 1-1.5, 1.5-5, 1.5-4.5, 1.5-4, 1.5-3.5, 1.5-3, 1.5-2.5, 1.5-2, 2-5, 2-4.5, 2-4, 2-3.5, 2-3, 2-2.5, 2.5-5, 2.5-4.5, 2.5-4, 2.5-3.5, 2.5-3, 3-5, 3-4.5, 3-4, 3-3.5, 3.5-5, 3.5-4.5, 3.5-4, 4-5, 4-4.5, or 4.5-5. In some aspects, the dentifrice composition is substantially free of, or does not include, a sweetener.

In some aspects, the dentifrice compositions comprise one or more whitening agents. In some aspects, the one or more whitening agents comprise titanium dioxide, hydrogen peroxide, sodium tripolyphosphates, or any combination thereof. In some aspects, the dentifrice compositions comprise one or more whitening agents in an amount of 0 wt. % or greater than 0 wt. %. In some aspects, the dentifrice compositions comprise one or more whitening agents in an amount of 2 wt. % or less. In some aspects, the dentifrice compositions comprise one or more whitening agents in an amount (wt. %) of 0-2, 0-1.5, 0-1, 0-0.5, 0-0.1, >0-2, >0-1.5, >0-1, >0-0.5, >0-0.1, 0.1-2, 0.1-1.5, 0.1-1, 0.1-0.5, 0.5-2, 0.5-1.5, 0.5-1, 1-2, 1-1.5, or 1.5-2. In some aspects, the dentifrice compositions do not contain a whitening agent.

In some aspects, the dentifrice compositions comprise one or more abrasives. In some aspects, the one or more abrasives comprise silica, zinc orthophosphate, sodium bicarbonate, alumina, calcium carbonate, calcium pyrophosphate, or any combination thereof. Although the glass compositions disclosed herein may have an abrasive effect in the dentifrice compositions, the glass compositions are not considered an abrasive herein for purposes of the amounts or inclusion of the one or more abrasives. In some aspects, the dentifrice compositions comprise one or more abrasives in an amount of 0 wt. % or greater than 0 wt. %. In some aspects, the dentifrice compositions comprise one or more abrasives in an amount of 20 wt. % or less. In some aspects, the dentifrice compositions comprise one or more abrasives in an amount (wt. %) of 0-20, 0-15, 0-10, 0-5, 0-1, 1-20, 1-15, 1-10, 1-5, 5-20, 5-15, 5-10, 10-20, 10-15, or 15-20. In some aspects, the dentifrice compositions do not contain an abrasive.

In some aspects, the dentifrice compositions may comprise other ingredients that contribute in some way to a desired dentifrice composition. For example, in some aspects, the dentifrice compositions can comprise one or more of a colorant, a fragrance, a medication, a preservative, a desensitizing agent (e.g., potassium salts such as potassium nitrate), a pH modifiers, a preservative, a tartar control agent, and so forth, or any combination thereof. In some aspects, the dentifrice compositions comprise one or more other ingredients in an amount of 0 wt. % or greater than 0 wt. %. In some aspects, the dentifrice compositions comprise one or more other ingredients in an amount of 10 wt. % or less. In some aspects, the dentifrice compositions comprise one or more other ingredients in an amount (wt. %) of 0-10, 0-9, 0-8, 0-7, 0-6, 0-5, 0-4, 0-3, 0-2, 0-1, 0-0.5, >0-10, >0-9, >0-8, >0-7, >0-6, >0-5, >0-4, >0-3, >0-2, >0-1, >0-0.5, 0.5-10, 0.5-9, 0.5-8, 0.5-7, 0.5-6, 0.5-5, 0.5-4, 0.5-3, 0.5-2, 0.5-1, 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10. The amounts of the one or more ingredients can be used to describe the amount of any one of the other ingredients, or can be used to describe the sum of any two or more of the other ingredients.

In some aspects, the dentifrice compositions comprise:
>0-20 wt. % of a glass composition, based on total weight of the matrix,
water, and
one or more of a humectant, a fluoride ion source, a flavoring, a surfactant, a sweetener, a whitening agent, an abrasive, or any combination thereof.

In some aspects, the dentifrice compositions comprise:
>0-20 wt. % of a glass composition, based on total weight of the matrix,
glycerin, and
one or more of a humectant, a fluoride ion source, a flavoring, a surfactant, a sweetener, a whitening agent, an abrasive, or any combination thereof.

In some aspects, disclosed is a method for making the matrix, comprising combining the glass composition with a carrier. In some aspects, the carrier is a toothpaste, mouthwash, gel, varnish, rinse, spray, ointment, salve, cream, bandage, polymer film, oral formulation, pill, capsule, or transdermal formulation.

In some aspects, disclosed is a method comprising applying the matrix to a bone or tooth. In some aspects, disclosed is a method comprising applying the matrix to a bone or tooth. In some aspects, this method is performed by an end user, for example, by way of brushing teeth with a toothpaste or swishing a mouth with a mouthwash. In some aspects, this method is performed by a professional, such as a doctor, a dentist, or other oral care professional, by way of applying the matrix to a bone or tooth, brushing a patient's teeth, directing a patient to swish their mouth with a mouthwash, applying the matrix to patient's teeth, and so forth.

In some aspects, the method of applying the matrix to a bone remineralizes the bone or tooth. In some aspects, the method of applying the matrix to a tooth treats caries of the tooth, treats dentin hypersensitivity of the tooth, or any combination thereof. In some aspects, the method of applying the matrix to a tooth remineralizes the tooth (e.g., by forming apatite in or on the tooth, such as in or on the enamel of the tooth). In some aspects, the method of applying the matrix to a tooth treats caries of the tooth, treats dentin hypersensitivity of the tooth, or any combination thereof, remineralizes the tooth (e.g., enamel) by forming apatite in or on the tooth (e.g., enamel). In some aspects, the method is facilitated by contact with saliva in a mouth (e.g., a patient's mouth).

Various aspects are contemplated herein, several of which are set forth in the paragraphs below. It is explicitly contemplated that any aspect or portion thereof can be combined to form a combination.

Aspect 1: A glass composition, comprising:
15-50 wt. % SiO₂,
25-60 wt. % CaO,
3-30 wt. % P₂O₅,
0.5-15 wt. % ZrO₂, and
>0-5 wt. % F,
wherein, when subjected to artificial saliva of Table 1A or Table 1B, the glass composition forms a bioactive crystalline phase.

Aspect 2: The glass composition of aspect 1, comprising:
25-45 wt. % SiO₂,
30-50 wt. % CaO,
5-25 wt. % P₂O₅,
2-10 wt. % ZrO₂, and
0.01-3 wt. % F.

Aspect 3: The glass composition of any one of aspects 1-2, or any preceding aspect, comprising >0-25 wt. % MgO.

Aspect 4: The glass composition of any one of aspects 1-3, or any preceding aspect, comprising >0-5 wt. % B₂O₃.

Aspect 5: The glass composition of any one of aspects 1-4, or any preceding aspect, comprising >0-5 wt. % Al₂O₃.

Aspect 6: The glass composition of any one of aspects 1-5, or any preceding aspect, comprising >0-10 wt. % Li₂O.

Aspect 7: The glass composition of any one of aspects 1-6, or any preceding aspect, comprising >0-10 wt. % Na₂O.

Aspect 8: The glass composition of any one of aspects 1-7, or any preceding aspect, comprising >0-10 wt. % K₂O.

Aspect 9: The glass composition of any one of aspects 1-8, or any preceding aspect, comprising >0-10 wt. % SrO.

Aspect 10: The glass composition of any one of aspects 1-9, or any preceding aspect, comprising >0-10 wt. % ZnO.

Aspect 11: The glass composition of any one of aspects 1-10, or any preceding aspect, comprising Li₂O+Na₂O+K₂O in an amount of 10 wt. % or less.

Aspect 12: The glass composition of any one of aspects 1-11, or any preceding aspect, comprising F+P₂O₅ in an amount of 5-30 wt. %.

Aspect 13: The glass composition of any one of aspects 1-12, or any preceding aspect, comprising MgO+CaO in an amount of 30-70 wt. %.

Aspect 14: The glass composition of any one of aspects 1-13, or any preceding aspect, comprising CaO+P₂O₅ in an amount of 40-70 wt. %.

Aspect 15: The glass composition of any one of aspects 1-14, or any preceding aspect, wherein the F is derived from calcium fluoride, magnesium fluoride, sodium fluoride, potassium fluoride, stannous fluoride, sodium monofluorophosphate, sodium difluorophosphate, or any combination thereof.

Aspect 16: The glass composition of any one of aspects 1-15, or any preceding aspect, wherein the glass composition is substantially free of, or does not contain, a crystalline phase.

Aspect 17: The glass composition of any one of aspects 1-16, or any preceding aspect, wherein, when subjected to artificial saliva of Table 1A or Table 1B, the bioactive crystalline phase comprises apatite.

Aspect 18: The glass composition of aspect 17, or any preceding aspect, wherein the apatite comprises hydroxyapatite, fluorapatite, carbonated apatite, or any combination thereof.

Aspect 19: The glass composition of any one of aspects 1-18, or any preceding aspect, wherein the artificial saliva has the composition of Table 1B.

Aspect 20: The glass composition of any one of aspects 1-19, or any preceding aspect, wherein the glass composition is in a form of particulates, microbeads, fibers or a combination thereof.

Aspect 21: A matrix comprising the glass composition of any one of aspects 1-20, or any preceding aspect, wherein:
the glass composition is attached to the matrix or mixed therein, and
the matrix includes at least one of: a toothpaste, mouthwash, gel, varnish, rinse, spray, ointment, salve, cream, bandage, polymer film, oral formulation, pill, capsule, or transdermal formulation.

Aspect 22: The matrix of aspect 21, or any preceding aspect, wherein the matrix is substantially free of, or does not include, a fluoride ion source.

Aspect 23: The matrix of aspect 21 or 22, or any preceding aspect, wherein the matrix is a dentifrice composition.

Aspect 24: The matrix of aspect 23, or any preceding aspect, wherein the dentifrice composition comprises:
>0-20 wt. % of the glass composition, based on total weight of the matrix,
water, and
one or more of a humectant, a fluoride ion source, a flavoring, a surfactant, a sweetener, a whitening agent, an abrasive, or any combination thereof.

Aspect 25: The matrix of aspect 23, or any preceding aspect, wherein the dentifrice composition comprises:
>0-20 wt. % of the glass composition, based on total weight of the matrix,
glycerin, and
one or more of a humectant, a fluoride ion source, a flavoring, a surfactant, a sweetener, a whitening agent, an abrasive, or any combination thereof.

Aspect 26: A method for making the matrix of any one of aspects 21-25, or any preceding aspect, comprising combining the glass composition with a carrier.

Aspect 27: A method comprising applying to a tooth the matrix of any one of aspects 21-26, or any preceding aspect.

Aspect 28: The method of aspect 27, or any preceding aspect, wherein the method remineralizes enamel of the tooth, treats caries of the tooth, treats dentin hypersensitivity of the tooth, or any combination thereof.

Aspect 29: A method for making the glass composition of any one of aspects 1-20, or any preceding aspect, comprising:
combining each component of the glass composition to form a mixture,
heating the mixture to a temperature of 1500° C. or less to form a melted mixture, and
cooling the melted mixture to form a cooled mixture.

Aspect 30: The method of aspect 29, or any preceding aspect, further comprising forming the cooled mixture into particulates, microbeads, fibers, or a combination thereof.

Aspect 31: A combination of any two or more of aspects 1-30, or any one or more portions thereof.

### Example

The following examples illustrate non-limiting aspects of the disclosure and are not intended to be limiting on the scope of the disclosure or claims.

Non-limiting examples of amounts of precursor oxides for forming the glass compositions are listed in Table 3.

TABLE 3

| Oxide (wt. %) | Comp. Ex. | 1 | 2 | 3 | 4 | 5 | 6 |
|---|---|---|---|---|---|---|---|
| SiO₂ | 45.0 | 33.45 | 33.3 | 31.4 | 33.4 | 30.4 | 31.4 |
| MgO | 0 | 4.5 | 4.5 | 4.5 | 4.5 | 4.5 | 4.5 |
| CaO | 24.5 | 42.5 | 42.5 | 42.5 | 42.5 | 42.5 | 42.5 |
| P₂O₅ | 6.0 | 15.5 | 15.5 | 17.5 | 15.5 | 18.5 | 17.5 |
| ZrO₂ | 0 | 4 | 4 | 4 | 4 | 4 | 4 |
| F⁻ | 0 | 0.05 | 0.2 | 0.1 | 0.1 | 0.1 | 0.1 |

| Oxide (wt. %) | 7 | 8 | 9 | 10 | 11 | 12 |
|---|---|---|---|---|---|---|
| SiO₂ | 33.4 | 35.4 | 42 | 41 | 40 | 38 |
| MgO | 4.5 | 4.5 | 4.5 | 4.5 | 4.5 | 4.5 |
| CaO | 42.5 | 42.5 | 42.5 | 42.5 | 42.5 | 42.5 |
| P₂O₅ | 15.5 | 13.5 | 6 | 6 | 8 | 10 |
| ZrO₂ | 4 | 4 | 4 | 4 | 4 | 4 |
| F⁻ | 0.1 | 0.1 | 1 | 2 | 1 | 1 |

The glass compositions of Table 3 may be melted at temperatures below 1500° C., or at temperatures below 1400° C., or at temperatures below 1200° C., thereby making it possible to melt in relatively small commercial glass tanks. The glass compositions disclosed herein (e.g., Table 3) exhibit significantly higher chemical durability and excellent bioactivity, as compared to currently available glasses, and can be in any form that is useful for the medical and dental processes disclosed. In some aspects, the compositions of Table 3 demonstrate significantly higher chemical durability and bioactivity over the Comparative Example (45S5 glass).

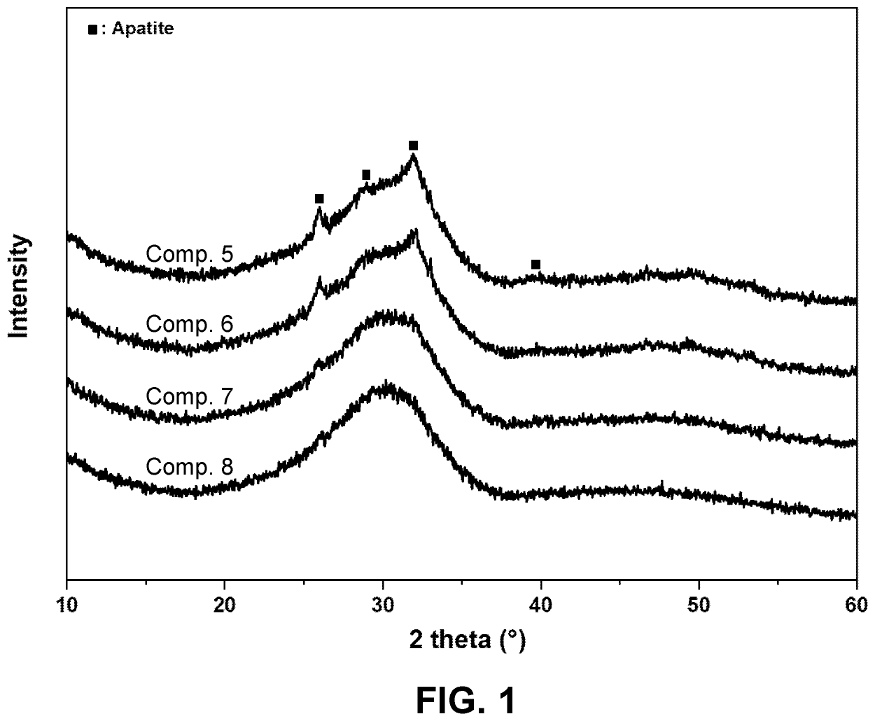

FIG. 1 illustrates powder x-ray diffraction (XRD) analysis of four example compositions prepared in accordance with the disclosures herein that have been soaked in artificial saliva for 14 days. The location of XRD peaks corresponding to apatite formation are shown in FIG. 1, indicating the formation of apatite in example compositions. Powder XRD analysis was conducted by preparing the samples by first drying and then grinding to a fine powder using a Rocklabs ring mill. The powder was then analyzed using a Bruker D4 Endeavor device equipped with a LynxEye™ silicon strip detector. X-ray scanning was conducted from 5° to 80° (2θ) for data collection.

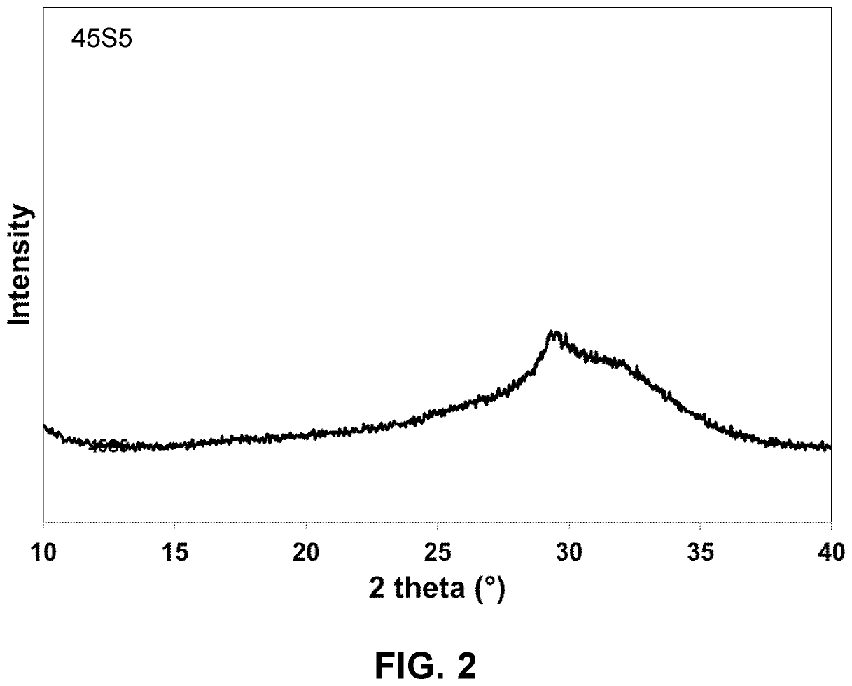

In contrast, FIG. 2 illustrates powder XRD analysis of the Comparative Example (45S5 Bioglass®) after soaking in artificial saliva for 61 days. Though a calcite (CaCO₃) phase started to form after 47 days soaking in artificial saliva (data not shown), the Comparative Example showed no hydroxyapatite formation even after 61 days in artificial saliva.

As specified by ASTM F1538-03, bioactive materials only refer to those that can form apatite in vitro or in vivo. The formation of calcite does not produce a direct bond with hard tissues due to its chemical difference from bone and teeth; thus, calcite is not regarded as bioactive. The inclusion of relatively high concentrations of CaO and P₂O₅ oxides, as well as F, in the example compositions facilitates the formation of an apatite phase in artificial saliva while the Comparative Examples composition (which has appreciably lower concentrations of CaO and P₂O₅ and no F) only forms a calcite phase. Due to its chemical similarity with hard tissues, such as bone and teeth, apatite can form a direct bond with hard tissue while calcite cannot.

Because the apatite phase forms in the example compositions without the inclusion of a fluoride ion source (i.e., beyond the F contained within the glass composition itself), there is no need to supply any external fluoride ion in addition to the glass composition (e.g., by way of typical fluoride ion sources present in oral care products, such as calcium fluoride, magnesium fluoride, sodium fluoride, potassium fluoride, stannous fluoride, sodium monofluorophosphate, sodium difluorophosphate, or any combination thereof). However, in some aspects, it still may be desirable to include such an external fluoride ion source in addition to the glass composition (i.e., which generally already contains F⁻).

As used herein, the term “and/or,” when used in a list of two or more items, means that any one of the listed items can be employed by itself, or any combination of two or more of the listed items can be employed. For example, if a composition is described as containing components A. B. and/or C, the composition can contain A alone; B alone; C alone; A and B in combination; A and C in combination; B and C in combination; or A, B, and C in combination.

References herein to the positions of elements (e.g., “top,” “bottom,” “above,” “below,” “first,” “second,” etc.) are merely used to describe the orientation of various elements in the FIGURES. It should be noted that the orientation of various elements may differ according to other exemplary embodiments, and that such variations are intended to be encompassed by the present disclosure. Moreover, these relational terms are used solely to distinguish one entity or action from another entity or action, without necessarily requiring or implying any actual such relationship or order between such entities or actions.

Modifications of the disclosure will occur to those skilled in the art and to those who make or use the disclosure. Therefore, it is understood that the embodiments shown in the drawings and described above are merely for illustrative purposes and not intended to limit the scope of the disclosure, which is defined by the following claims, as interpreted according to the principles of patent law, including the doctrine of equivalents.

It will be understood by one having ordinary skill in the art that construction of the described disclosure, and other components, is not limited to any specific material. Other exemplary embodiments of the disclosure disclosed herein may be formed from a wide variety of materials, unless described otherwise herein.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

As utilized herein, “optional,” “optionally,” or the like are intended to mean that the subsequently described event or circumstance can or cannot occur, and that the description includes instances where the event or circumstance occurs and instances where it does not occur. As used herein, the indefinite articles “a,” “an,” and the corresponding definite article “the” mean “at least one” or “one or more,” unless otherwise specified. It also is understood that the various features disclosed in the specification and the drawings can be used in any and all combinations.

With respect to the use of substantially any plural and/or singular terms herein, those having skill in the art can translate from the plural to the singular and/or from the singular to the plural as is appropriate to the context and/or application. The various singular/plural permutations may be expressly set forth herein for the sake of clarity.

Unless otherwise specified, all compositions are expressed in terms of as-batched weight percent (wt. %). As will be understood by those having ordinary skill in the art, various melt constituents (e.g., silicon, alkali- or alkaline-based, boron, etc.) may be subject to different levels of volatilization (e.g., as a function of vapor pressure, melt time and/or melt temperature) during melting of the constituents. As such, the as-batched weight percent values used in relation to such constituents are intended to encompass values within ±0.5 wt. % of these constituents in final, as-melted articles. With the forgoing in mind, substantial compositional equivalence between final articles and as-batched compositions is expected.

It will be apparent to those skilled in the art that various modifications and variations can be made without departing from the spirit or scope of the claimed subject matter. Accordingly, the claimed subject matter is not to be restricted except in light of the attached claims and their equivalents.

What is claimed is:

1. A glass composition, comprising:
15-50 wt. % SiO₂,
25-60 wt. % CaO,
3-30 wt. % P₂O₅,
0.5-15 wt. % ZrO₂, and
>0-5 wt. % F,
wherein, when subjected to artificial saliva of Table 1A or Table 1B, the glass composition forms a bioactive crystalline phase.

2. The glass composition of claim 1, comprising:
25-45 wt. % SiO₂,
30-50 wt. % CaO,
5-25 wt. % P₂O₅,
2-10 wt. % ZrO₂, and
0.01-3 wt. % F.

3. The glass composition of claim 1, comprising at least one of:
>0-25 wt. % MgO;
>0-5 wt. % B₂O₃;
>0-5 wt. % Al₂O₃;
>0-10 wt. % Li₂O;
>0-10 wt. % Na₂O;
>0-10 wt. % K₂O;
>0-10 wt. % SrO;
>0-10 wt. % ZnO;

4. The glass composition of claim 1, comprising Li₂O+Na₂O+K₂O in an amount of 10 wt. % or less.

5. The glass composition of claim 1, comprising at least one of:
F+P₂O₅ in an amount of 5-30 wt. %;
MgO+CaO in an amount of 30-70 wt. %;
CaO+P₂O₅ in an amount of 40-70 wt. %;

6. The glass composition of claim 1, wherein the F is derived from calcium fluoride, magnesium fluoride, sodium fluoride, potassium fluoride, stannous fluoride, sodium monofluorophosphate, sodium difluorophosphate, or any combination thereof.

7. The glass composition of claim 1, wherein the glass composition is substantially free of a crystalline phase.

8. The glass composition of claim 1, wherein, when subjected to artificial saliva of Table 1A or Table 1B, the bioactive crystalline phase comprises apatite.

9. The glass composition of claim 8, wherein the apatite comprises hydroxyapatite, fluorapatite, carbonated apatite, or any combination thereof.

10. The glass composition of claim 1, wherein the glass composition is in a form of particulates, microbeads, fibers or a combination thereof.

11. A matrix comprising the glass composition of claim 1, wherein:
the glass composition is attached to the matrix or mixed therein, and
the matrix includes at least one of: a toothpaste, mouthwash, gel, varnish, rinse, spray, ointment, salve, cream, bandage, polymer film, oral formulation, pill, capsule, or transdermal formulation.

12. The matrix of claim 11, wherein the matrix is substantially free of a fluoride ion source.

13. The matrix of claim 11, wherein the matrix is a dentifrice composition.

14. The matrix of claim 13, wherein the dentifrice composition comprises:
>0-20 wt. % of the glass composition, based on total weight of the matrix,
water, and
one or more of a humectant, a fluoride ion source, a flavoring, a surfactant, a sweetener, a whitening agent, an abrasive, or any combination thereof.

15. The matrix of claim 13, wherein the dentifrice composition comprises:
>0-20 wt. % of the glass composition, based on total weight of the matrix,
glycerin, and
one or more of a humectant, a fluoride ion source, a flavoring, a surfactant, a sweetener, a whitening agent, an abrasive, or any combination thereof.

16. A method for making the matrix of claim 11, comprising combining the glass composition with a carrier.

17. A method comprising applying to a tooth the matrix of claim 1.

18. The method of claim 17, wherein the method remineralizes enamel of the tooth, treats caries of the tooth, treats dentin hypersensitivity of the tooth, or any combination thereof.

19. A method for making the glass composition of claim 1, comprising:
combining each component of the glass composition to form a mixture,
heating the mixture to a temperature of 1500° C. or less to form a melted mixture, and
cooling the melted mixture to form a cooled mixture.

20. The method of claim 19, further comprising forming the cooled mixture into particulates, microbeads, fibers, or a combination thereof.
